(19) United States
(12) Patent Application Publication
(10) Pub. No.: US 2013/0059378 A1
(43) Pub. Date: Mar. 7, 2013

(54) HUMAN EPIDERMIS-DERIVED MESENCHYMAL STEM CELL-LIKE PLURIPOTENT CELLS AND PREPARATION METHOD THEREOF

(71) Applicants: Bing Huang, Guangzhou (CN); Jian Ge, Guangzhou (CN)

(72) Inventors: Bing Huang, Guangzhou (CN); Jian Ge, Guangzhou (CN)

(21) Appl. No.: 13/695,293

(22) PCT Filed: Oct. 16, 2010
(86) PCT No.: PCT/CN10/77808

§ 371 Date: Oct. 30, 2012

(30) Foreign Application Priority Data
Sep. 13, 2010 (CN) 201010282388.0

(51) Int. Cl. C12N 5/071

(52) U.S. Cl. 435/371

(57) ABSTRACT

The invention discloses a cell culture medium, human epidermis-derived mesenchymal stem cell-like pluripotent cells and a preparation method thereof. The culture medium is formed by adding fetal bovine serum, hbFGF, hSCF, non-essential amino acids, L-glutamine and gentamicin into a DMEM culture medium with the glucose content of 0.5-5 g/L, wherein every 100 mL of the final culture medium contains 10-25% by volume percent of fetal bovine serum, 100-4000 ng of the hbFGF, 10-2000 ng of hSCF, 0.1-2 mL of 100× non-essential amino acids, 0.1-2 mL of PBS solution containing 3% by mass percent of L-glutamine and 1000-8000 U of gentamicin. A human epidermis-derived mesenchymal stem cell-like pluripotent cell strain is prepared by culturing human epidermal cells in the culture medium, digesting with digestive juice, removing undigested human epidermal cells, collecting digested mesenchymal stem cell-like cells and performing in-vitro culture and passage.

### BACKGROUND OF THE INVENTION

1. Field of the Invention

The invention relates to the technical field of biological cells and specifically relates to a cell culture medium capable of obtaining human epidermis-derived mesenchymal stem cell-like pluripotent cells by separating from human epidermal cells, as well as the human epidermis-derived mesenchymal stem cell-like pluripotent cells obtained by separation and a preparation method thereof.

2. Description of the Prior Art

Stem cells include embryonic stem cells and adult stem cells. Due to the reasons of difficult material sources, ethical issues, heterogeneous (allogeneic) immunological rejection and the like, the embryonic stem cells are difficult to apply clinically. The adult stem cells exist in various tissues of adults. Due to the extensive material sources, the adult stem cells can implement autologous cell transplantation and further have extensive application prospects in clinical treatment of diseases. At present, the adult stem cells which have been separated successfully at home and abroad comprise mesenchymal stem cells, epidermal stem cells, neural stem cells, adipose-derived stem cells, islet stem cells and the like. However, the adult stem cells which have been reported at present are slow in growth or limited in amplification generation number, so that the adult stem cells can not obtain a huge number of cells required for clinical treatment of the diseases within a short period of time (three months). This is also one of bottlenecks for hindering the clinical application of the adult stem cells at present.

### SUMMARY OF THE INVENTION

The first purpose of the invention is to provide a cell culture medium capable of obtaining human epidermis-derived mesenchymal stem cell-like pluripotent cells by isolation from human epidermal cells, and the cell culture medium can also be used for passage culturing of the human epidermis-derived mesenchymal stem cell-like pluripotent cells.

The cell culture medium capable of obtaining the human epidermis-derived mesenchymal stem cell-like pluripotent cells by isolating from the human epidermal cells is formed by adding fetal bovine serum, human basic fibroblast growth factor (hbFGF), human stem cell factor (hSCF), non-essential amino acids, L-glutamine and gentamicin into a DMEM culture medium with glucose content of 0.5-5 g/L, wherein every 100 mL of the final culture medium contains 10-25% by volume percent of fetal bovine serum, 100-4000 ng of hbFGF, 10-2000 ng of hSCF, 0.1-2 mL of 100× non-essential amino acids, 0.1-2 mL of PBS solution containing 3% by mass percent of L-glutamine and 1000-8000 U of gentamicin.

The DMEM culture medium of the invention is the publicly known culture medium, and all glucose, fetal bovine serum, hbFGF, hSCF, the 100× non-essential amino acids, L-glutamine, the PBS solution and gentamicin are well-know products, which can be prepared according to prior arts or purchased from reagent companies.

A preparation method of the cell culture medium capable of obtaining the human epidermis-derived mesenchymal stem cell-like pluripotent cells by isolating from the human epidermal cells comprises the following steps: adding glucose into the DMEM culture medium, and uniformly stirring to the final concentration of glucose with 0.5-5 g/L so as to form a primary basal culture medium; and then adding fetal bovine serum, hbFGF, hSCF, the non-essential amino acids, L-glutamine and gentamicin into the primary basal culture medium, and uniformly stirring so as to prepare the cell culture medium capable of obtaining the human epidermis-derived mesenchymal stem cell-like pluripotent cells by separating from the human epidermal cells, wherein every 100 mL of the final culture medium contains 10-25% by volume percent of fetal bovine serum, 100-4000 ng of hbFGF, 10-2000 ng of hSCF, 0.1-2 mL of 100× non-essential amino acids, 0.1-2 mL of PBS solution containing 3% by mass percent of L-glutamine and 1000-8000 U of gentamicin, wherein the PBS solution containing 3% by mass percent of L-glutamine is prepared by adding 3 g of L-glutamine in every 100 mL of PBS solution.

The second purpose of the invention is to provide a method for preparing the human epidermis-derived mesenchymal stem cell-like pluripotent cells by utilizing the cell culture medium of the present invention and the human epidermis-derived mesenchymal stem cell-like pluripotent cells prepared by the method.

The preparation method of the human epidermis-derived mesenchymal stem cell-like pluripotent cells of the invention comprises the following steps:

a) culturing the human epidermal cells in the above-mentioned cell culture medium; and

b) digesting the human epidermal cells with digestive juice, removing undigested human epidermal cells, and collecting digested mesenchymal stem cell-like cells so as to get the human epidermis-derived mesenchymal stem cell-like pluripotent cells.

The third purpose of the invention is to provide a passage culture method of the human epidermis-derived mesenchymal stem cell-like pluripotent cells, since the human epidermis-derived mesenchymal stem cell-like pluripotent cells can be passage cultured in-vitro. The method comprises the following step: performing the passage culture by using the cell culture medium of the present invention as a passage culture medium at 36±1° C. with 5±1% CO₂ and 90-100% humidity. The human epidermis-derived mesenchymal stem cell-like pluripotent cell strain is therefore obtained. The human epidermis-derived mesenchymal stem cell-like pluripotent cells of the present invention can grow fast and can be passed according to a proportion of 1:3 every 1-4 days. More than 30 generations can be passed within three months, and at least 2×10¹⁶ new adult stem cells can be obtained from 3×10⁴ cells in every clinically abandoned skin specimen within three months.

A preferred method for obtaining the human epidermal cells comprises the following steps: taking foreskin or other skin tissues, and digesting them with type II histolytic enzyme to obtain epidermis tissue, cutting the epidermis tissue into pieces, and further digesting it with trypsin to obtain the epidermal cells.

The human epidermal cells are cultured in the cell culture medium under the preferred culture conditions that the temperature is 36±1° C. CO₂ accounts for 5±1% and the humidity is 90-100%, and the cell culture medium is changed every 2-3 days.

Preferably, the step b) comprises the following steps: when the cells reach 60-90% confluence, digesting the human epidermal cells with 0.25% trypsin/0.02% wt EDTA (1:1), removing the undigested human epidermal cells and collecting the digested mesenchymal stem cell-like cells so as to get the human epidermis-derived mesenchymal stem cell-like pluripotent cells.

Staining smears were prepared from the human epidermis-derived mesenchymal stem cell-like pluripotent cells of the invention (as shown in FIG. 1 and FIG. 2) through a conventional method for performing karyotype check, and the check result was that a karyotype had 46 chromosomes of a normal man respectively, and banding patterns were normal (as shown in FIG. 3 and FIG. 4).

Surface markers of the human epidermis-derived mesenchymal stem cell-like pluripotent cells of the present invention were detected by using a flow cytometry and an immunohistochemistry process (a detection method was of a publicly known technology, a kit could be provided by a commercial reagent company and the detection was implemented according to the technical scheme provided by the reagent company), and the specific markers of mesenchymal stem cells and neural precursor cells included CD73 (93.5-99.8%), CD90 (63.4-99.7%), CD105 (20.3-92.8%), Vimentin (51.3-96.7%) and Nestin (74.2-98.4%). As shown in FIG. 5, positive cells were brown or brownish black; and markers with no expression or low expression of neural terminal cells, epidermal cells, blood cells and vascular endothelial cells included β-III tubulin (0-18.7%), MAP-2 (0-3.2%), GFAP (0-7.2%), CK19 (0-1.0%), CD34 (0-1.3%), CD45 (0-0.5%) and CD3 (0-1.1%), CD19 (0-0.5%), CD16 (0-0.7%), CD4 (0-0.5%), CD8 (0-0.7%), CD31 (0-5.8%), VEGF-R2 (0-5.5%) and CD10 (0.2-18.2%).

The human epidermis-derived mesenchymal stem cell-like pluripotent cell strain of the invention was cultured in the serum-free neural stem cell culture medium (purchased from GIBCO Company) for 6 days, and then cultured by the neural stem cell culture medium (purchased from GIBCO Company) in which fetal bovine serum is added till the final volume percent achieves 10% for 7 days, and microscopical examination found that the cells had formed neural cell-like slender processus (as shown in FIG. 6). The surface markers of the cells were detected by a laser confocal microscopic observation technology (a detection method was of a publicly known technology, a kit could be provided by a commercial reagent company and the detection was implemented according to the technical scheme provided by the reagent company). The specific markers MAP-2 and β-III tubulin expressed in neural cells were found, and the positive cells showed red fluorescence (as shown in FIG. 7, in which the blue is cell nucleus Hochest staining, the red is labeling red fluorescence monoclonal antibody staining, and as FIG. 7 is a black-and-white diagram, blue cell nuclei are oval, and the red fluorescence is dot-like). This shows that the human epidermis-derived mesenchymal stem cell-like pluripotent cell strain of the present invention can be differentiated into neural-like cells.

The human epidermis-derived mesenchymal stem cell-like pluripotent cell strain of the invention was cultured by the immune cell culture medium (purchased from GIBCO Company) for 7 days, and then subjected to a microscopical examination and a morphologic observation. It was found that some secretory vesicles were formed in the cells, and a large number of circular vesicles with strong light refraction was existed inside and outside the cells (as shown in FIG. 8). The increase in the quantity of the specific markers expressed in immune cells was detected by a flow cytometry, such as CD19 (1.7-11.3%), CD16 (1.3-5.4%), CD4 (7.3-14.5%) and CD8 (4.2-8.1%). This shows that the human epidermis-derived mesenchymal stem cell-like pluripotent cell strain of the invention can be differentiated into immune cell-like cells.

Each embryo of the blastocoeles of mice was injected 4-8 human epidermis-derived mesenchymal stem cell-like pluripotent cells of the invention for making a chimeric mice (a conventional making method), and the detection was performed by using a PCR (polymerase chain reaction) technology, a human Y chromosome in-situ hybridization technology and an anti-human monoclonal antibody immunofluorescence laser confocal microscopic observation technology (a detection method was of a publicly known technology, a kit could be provided by a commercial reagent company and the detection was implemented according to the technical scheme provided by the reagent company). It was found that the human Y chromosomes, human CD 19 and other proteins were existed in the blood of the chimeric mice. As shown in FIG. 9, which shows a PCR electrophoretogram, the detection via the PCR technology reveals that human Y chromosome marking genes, namely SRY genes (No.10 and No.11 lanes) were existed in the blood of the No. 10 and the No. 11 chimeric mice (about 5.5 months old). As shown in FIG. 10, which shows a detection chart via a human Y chromosome in-situ hybridization technology and an anti-human monoclonal antibody immunofluorescence laser confocal microscopic observation technology), it was found that the human Y chromosomes (shown by red points in FIG. 10A and FIG. 10D, which are pointed by arrows in FIG. 10A) and the human marking proteins CD19 (shown by green fluorescent dots in FIG. 10B and FIG. 10D) were existed in the blood of the No. 10 chimeric mouse (about 5.5 months old). This shows that the human epidermis-derived mesenchymal stem cell-like pluripotent cells of the invention can survive, migrate and differentiate in the chimeric mice within 5 months. The detection (a conventional method) via the flow cytometry and transplantation experiments in immune-deficient mice found that HLA-DR (0-1.3%) was of low expression or no expression, HLA-I (0.1-61.1%) was of low expression or moderate expression. It is also found that no tumors were in the mice after 3 months of an intraperitoneal injection and a subcutaneous injection of about 1×10⁷ cells into each SCID mouse. This indicates that the human epidermis-derived mesenchymal stem cell-like pluriotent cells of the invention can provide good biological safety.

In summary, the human epidermis-derived mesenchymal stem cell-like pluripotent cells of the invention have mesenchymal stem cell-like phenotype and normal karyotype. The human epidermis-derived mesenchymal stem cell-like pluripotent cells can express the specific markers of the mesenchymal stem cells and the neural precursor cells and realize no expression or low expression of the specific markers of the neural terminal cells, the epidermal cells, the blood cells and the vascular endothelial cells. The human epidermis-derived mesenchymal stem cell-like pluripotent cells can differentiate into the neural cell-like cells and the immune cell-like cells and have good biological safety.

By using the isolation method provided in the present invention, the human epidermis-derived mesenchymal stem cell-like pluripotent cells can be obtained by isolating the human epidermal cells which are grown in the cell culture medium provided by the present invention. The human epidermis-derived mesenchymal stem cell-like pluripotent cells have good biological safety, and are able to differentiating into the neural cell-like cells and the immune cell-like cells, providing a application of potential in repairing injuries by a nervous system, treating diseases by an immune system and constructing heterogeneous organs. The human epidermis-derived mesenchymal stem cell-like pluripotent cells provided in the present invention can grow fast. At least 30 generations can be passed within a short period of time (within 3 months) under the in-vitro passage culture conditions, and at least 2×10¹⁶ cells can be finally obtained from about 3×10⁴ cells of clinically abandoned skin specimen, thereby meeting the demands for clinical treatment of diseases and construction of tissue engineering organs.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 and FIG. 2 are optical morphological diagrams of the human epidermis-derived mesenchymal stem cell-like pluripotent cell strain, which show similar phenotypes to the mesenchymal stem cells;

FIG. 3 and FIG. 4 are karyotype diagram of human epidermis-derived mesenchymal stem cell-like pluripotent cell strain of the present invention;

FIG. 5 is a diagram showing that the human epidermis-derived mesenchymal stem cell-like pluripotent cells of the present invention express the specific markers of mesenchymal stem cells and neural precursor cells, wherein the diagram is provided by a flow cytometry and an immunohistochemistry process;

FIG. 6 is a diagram showing that the human epidermis-derived mesenchymal stem cell-like pluripotent cells of the present invention differentiate into neural cell-like cells, wherein the diagram is observed by a microscope;

FIG. 7 is a diagram showing the expression of specific markers of neural cells, MAP-2 and β-III tubulin, which are detected by a laser confocal microscopic; FIG. 7A shows a cell nucleus Hochest staining (blue), FIG. 7B shows a red fluorescence labeling monoclonal antibody staining which reveals cell bodies, and FIG. 7C shows a superposition diagram of FIG. 7A and FIG. 7B;

FIG. 8 is a diagram showing that the human epidermis-derived mesenchymal stem cell-like pluripotent cells of the present invention differentiate into immune cell-like cells, wherein the diagram is observed by a microscope;

FIG. 9 is a PCR electrophoretogram of the blood from the chimeric mice, wherein M: Marker, H₂O: water, N: normal mouse peripheral blood (negative control), 6: peripheral blood from No. 6 chimeric mouse, 7: peripheral blood from No. 7 chimeric mouse, 8: peripheral blood from No. 8 chimeric mouse, 9: peripheral blood from No. 9 chimeric mouse, 10: peripheral blood from No. 10 chimeric mouse, 11: peripheral blood from No. 11 chimeric mouse and P: human epidermis-derived mesenchymal stem cell-like pluripotent cells (positive control);

FIG. 10 is a diagram showing the blood of chimeric mice, detected by the human Y chromosome in-situ hybridization technology and the anti-human monoclonal antibody immunofluorescence confocal microscopic observation technology; FIG. 10A shows the diagram of human Y chromosome in-situ hybridization (red points represent human Y chromosomes), FIG. 10B shows the diagram of a green fluorescence-labeled human CD19 monoclonal antibody observed by a confocal microscope (green dots represent human CD 19 proteins), FIG. 10C shows the cell nucleus Hochest staining (blue) and FIG. 10D shows a superposition diagram of FIG. 10A, FIG. 10B and FIG. 10C. After superposition, it is shown that the human Y chromosomes are positioned in the cell nuclei and the human CD19 proteins are positioned on the cell body.

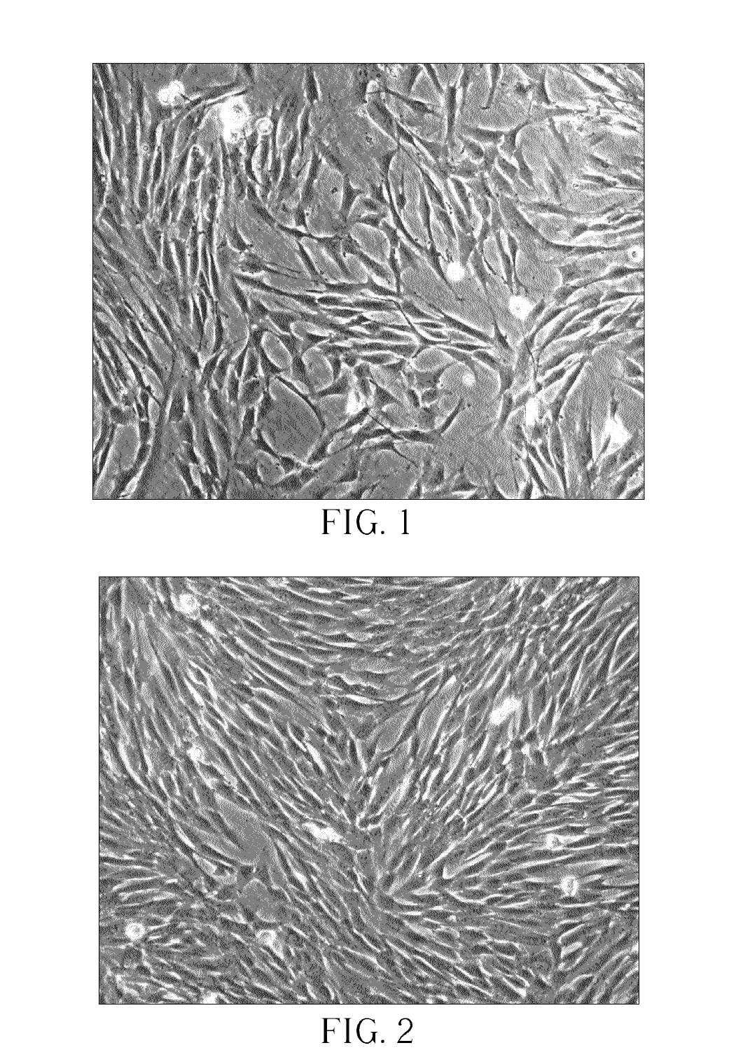

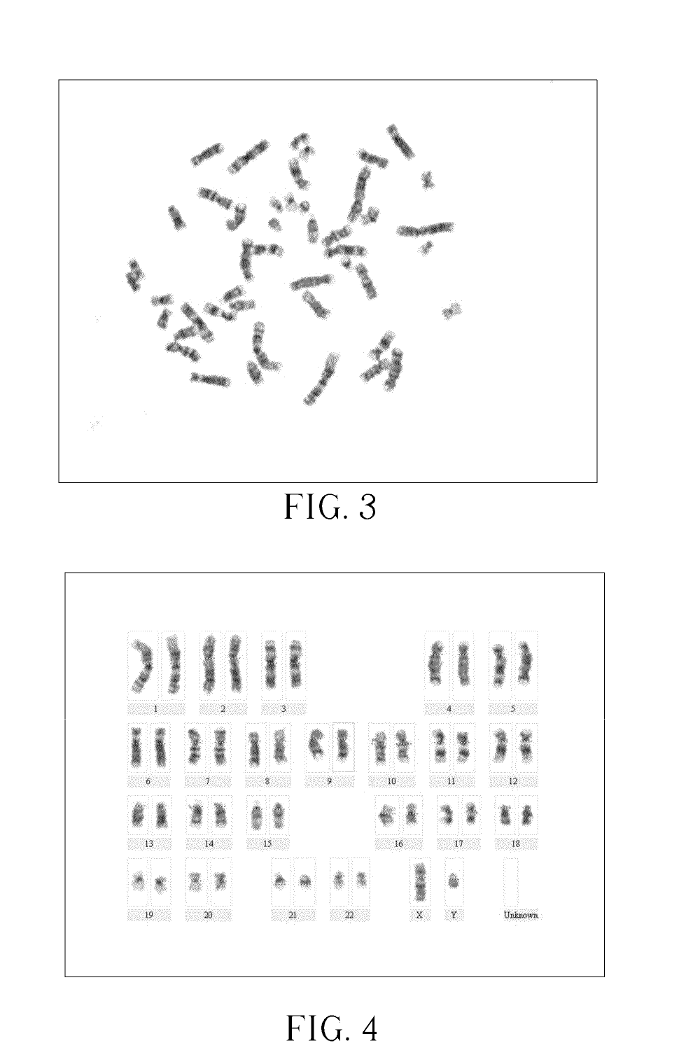

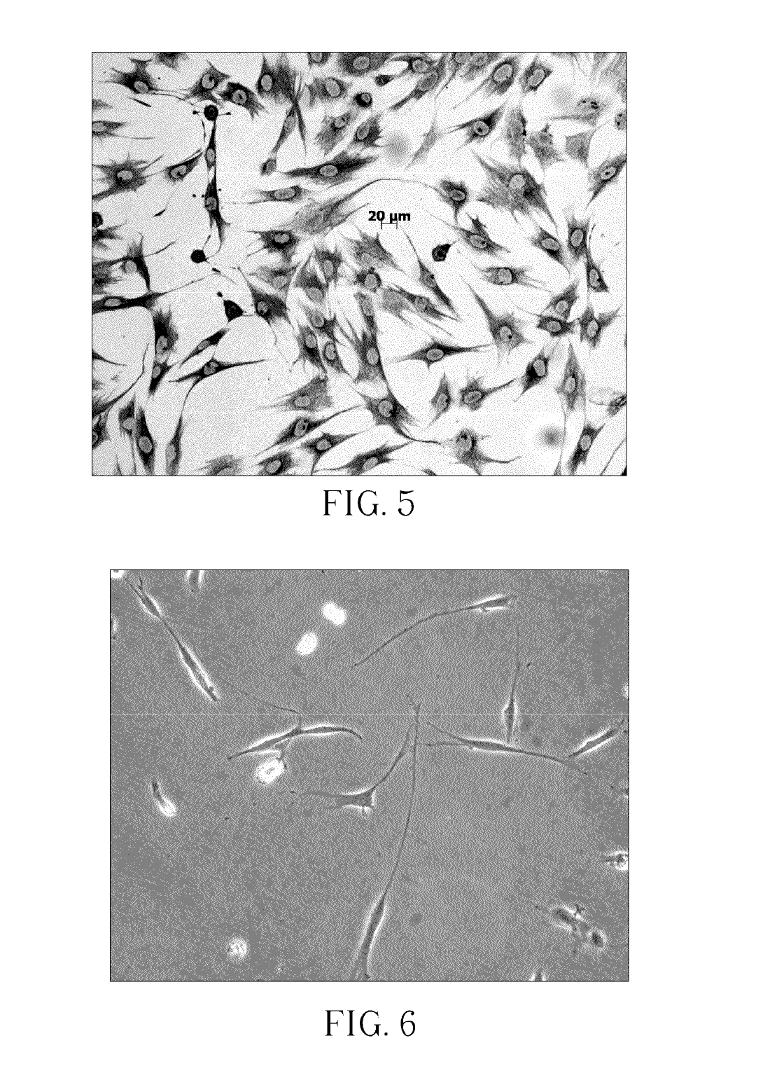

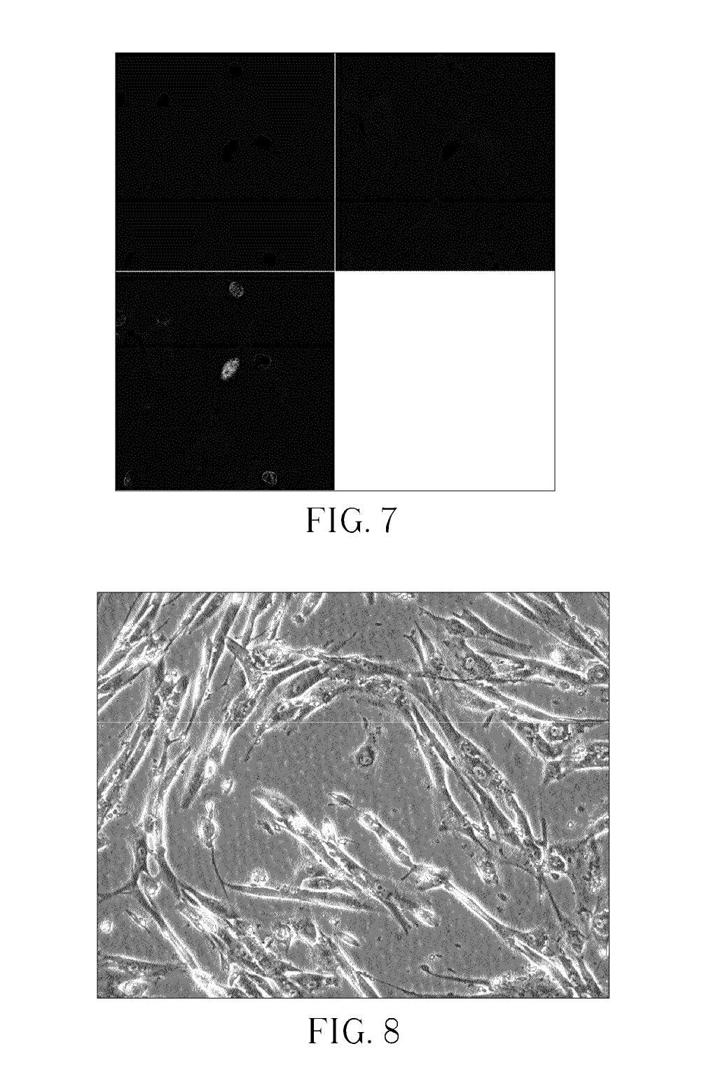

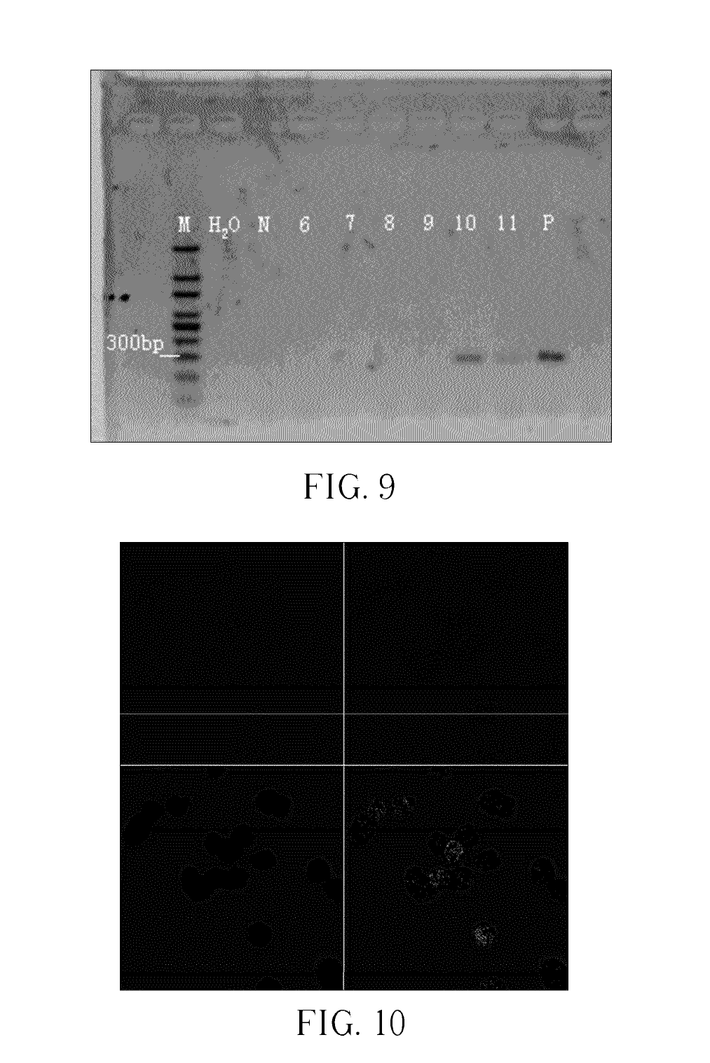

### DETAILED DESCRIPTION

The following embodiments are used for further describing the invention rather than limiting the invention.

### Embodiment 1

### 1. Preparation of Cell Culture Medium

The culture medium was prepared by adding fetal bovine serum, hbFGF, hSCF, non-essential amino acids, L-glutamine and gentamicin into a DMEM culture medium with the glucose content of 0.5 g/L, wherein every 100 mL of the final culture medium contains 10% by volume percent of fetal bovine serum, 4000 ng of hbFGF, 10 ng of hSCF, 1 mL of 100× non-essential amino acids, 2 mL of PBS solution containing 3% by mass percent of L-glutamine and 1000 U of gentamicin. The 100× non-essential amino acids were purchased from GIBCO Company and the article number is 11140.

### 2. Obtaining of Epidermal Tissue and Epidermal Cells

Clinically abandoned foreskin tissue was taken and soaked in PBS solution containing 1000 U of gentamicin per mL, and then preserved at the temperature of 4° C. within 4 hours. After fully washed with the PBS to remove the blood cells, the tissue was then cut off to remove the subcutaneous loose connective tissue, followed by soaking and digesting with type II histolytic enzyme (2 U/mL) overnight so as to obtain the epidermis tissue. The epidermis tissue was cut into pieces and then digested with 0.25% by mass percent of trypsin for 30 minutes to obtain the epidermal cells. Next, the epidermis cells were washed with PBS centrifuged to remove the digestive enzyme and re-suspending in the cell culture medium provided in the present embodiment.

### 3. Culture of the Epidermal Cells

The epidermal cells obtained from each specimen were harvested into 3-4 T25 culture flasks containing the cell culture medium provided by the present embodiment, cultured in an incubator at 36±1° C. with 5±1% CO₂ and 90-100% humidity. The cell culture medium was changed every 2-3 days.

### 4. Screening and Enrichment of Human Epidermis-Derived Mesenchymal Stem Cell-Like Pluripotent Cells

When the cells reached 60-90% confluence, the cultures were digested with 0.25% trypsin/0.02% wt EDTA (1:1). The digested mesenchymal stem cell-like cells were collected and undigested cells were discarded. The collected mesenchymal stem cell-like cells were centrifuged at 1000-1500 rpm for 5 minutes and the supernatant was discarded. The cells were re-suspended by using the cell culture medium provided in the present embodiment. Collected cells were the human epidermis-derived mesenchymal stem cell-like pluripotent cells in the invention. The cells were further counted.

s 5. In-Vitro Culture of Human Epidermis-Derived Mesenchymal Stem Cell-Like Pluripotent Cell Strain

The well-counted human epidermis-derived mesenchymal stem cell-like pluripotent cells were inoculated into T25 culture flasks containing the cell culture medium provided by the present embodiment to about 1×10⁵/mL, cultured in the incubator under at 36±1° C. with 5±1% CO₂ and 90-100% humidity, and passed to a proportion of 1:3 every 1-4 days till over 30 generations. The human epidermis-derived mesenchymal stem cell-like pluripotent cell strain is therefore obtained. The cells obtained from each clinically abandoned skin specimen can be passed above 30 generations within 3 months, and at least 2×10¹⁶ new human epidermis-derived mesenchymal stem cell-like pluripotent cells can be finally obtained from about 3×10⁴ cells.

By detecting of the human epidermis-derived mesenchymal stem cell-like pluripotent cell strain provided by the present embodiment, it was found that the cell strain has mesenchymal stem cell-like phenotype and normal karyotype, and is able to express specific markers of mesenchymal stem cells and neural precursor cells, including CD73, CD90, CD105, Vimentin and Nestin, and realize no expression or low expression of the specific markers of neural terminal cells, epidermal cells, blood cells and vascular endothelial cells, including β-III tubulin, MAP-2, GFAP, CK19, CD34, CD45, CD3, CD19, CD16, CD4, CD8, CD31, VEGF-R2 and CD10. The cell strain can differentiate into neural cell-like cells and immune cell-like cells, and realize low expression or no expression of HLA-DR and low expression or moderate expression of HLA-I. After 3 months of intraperitoneal injection and subcutaneous injection of about 1×10⁷ cells into each SCID mouse, no tumors were found in the mice. This indicates that the human epidermis-derived mesenchymal stem cell-like pluripotent cell strain in the present invention can provide good biological safety.

### Embodiment 2

### 1. Preparation of Cell Culture Medium

The culture medium was prepared by adding fetal bovine serum, hbFGF, hSCF, non-essential amino acids, L-glutamine and gentamicin into a DMEM culture medium with the glucose content of 5 g/L, wherein every 100 mL of the final culture medium contains 25% by volume percent of fetal bovine serum, 100 ng of hbFGF, 2000 ng of hSCF, 2 mL of 100× non-essential amino acids, 0.1 mL of PBS solution containing 3% by mass percent of L-glutamine and 8000 U of gentamicin. The 100× non-essential amino acids were purchased from GIBCO Company and the article number is 11140.

### 2. Obtaining of Epidermal Tissue and Epidermal Cells

Clinically abandoned foreskin tissue was taken and soaked in PBS solution containing 1000 U of gentamicin per mL, and then preserved at the temperature of 4° C. within 4 hours. After fully washed with the PBS to remove the blood cells, the tissue was then cut off to remove the subcutaneous loose connective tissue, followed by soaking and digesting with type II histolytic enzyme (2 U/mL) overnight so as to obtain the epidermis tissue. The epidermis tissue was cut into pieces and then digested with 0.25% by mass percent of trypsin for 30 minutes to obtain the epidermal cells. Next, the epidermis cells were washed with PBS centrifuged to remove the digestive enzyme and re-suspending in the cell culture medium provided in the present embodiment.

### 3. Culture of the Epidermal Cells

The epidermal cells obtained from each specimen were harvested into 3-4 T25 culture flasks containing the cell culture medium provided by the present embodiment, cultured in an incubator at 36±1° C. with 5±1% CO₂ and 90-100% humidity. The cell culture medium was changed every 2-3 days.

### 4. Screening and Enrichment of Human Epidermis-Derived Mesenchymal Stem Cell-Like Pluripotent Cells:

When the cells reached 60-90% confluence, the cultures were digested with 0.25% trypsin/0.02% wt EDTA (1:1). The digested mesenchymal stem cell-like cells were collected and undigested cells were discarded. The collected mesenchymal stem cell-like cells were centrifuged at 1000-1500 rpm for 5 minutes and the supernatant was discarded. The cells were re-suspended by using the cell culture medium provided in the present embodiment. Collected cells were the human epidermis-derived mesenchymal stem cell-like pluripotent cells in the invention. The cells were further counted.

### 5. In-Vitro Cculture of Human Epidermis-Derived Mesenchymal Stem Cell-Like Pluripotent Cell Strain

The well-counted human epidermis-derived mesenchymal stem cell-like pluripotent cells were inoculated into T25 culture flasks containing the cell culture medium provided by the present embodiment to about 1×10⁵/mL, cultured in the incubator under at 36±1° C. with 5±1% CO₂ and 90-100% humidity, and passed to a proportion of 1:3 every 1-4 days till over 30 generations. The human epidermis-derived mesenchymal stem cell-like pluripotent cell strain is therefore obtained. The cells obtained from each clinically abandoned skin specimen can be passed above 30 generations within 3 months, and at least 2×10¹⁶ new human epidermis-derived mesenchymal stem cell-like pluripotent cells can be finally obtained from about 3×10⁴ cells.

By detecting of the human epidermis-derived mesenchymal stem cell-like pluripotent cell strain provided by the present embodiment, it was found that the cell strain has mesenchymal stem cell-like phenotype and normal karyotype, and is able to express specific markers of mesenchymal stem cells and neural precursor cells, including CD73, CD90, CD105, Vimentin and Nestin, and realize no expression or low expression of the specific markers of neural terminal cells, epidermal cells, blood cells and vascular endothelial cells, including β-III tubulin, MAP-2, GFAP, CK19, CD34, CD45, CD3, CD19, CD16, CD4, CD8, CD31, VEGF-R2 and CD10. The cell strain can differentiate into neural cell-like cells and immune cell-like cells, and realize low expression or no expression of HLA-DR and low expression or moderate expression of HLA-I. After 3 months of intraperitoneal injection and subcutaneous injection of about 1×10⁷ cells into each SCID mouse, no tumors were found in the mice. This indicates that the human epidermis-derived mesenchymal stem cell-like pluripotent cell strain in the present invention can provide good biological safety.

### Embodiment 3

### 1. Preparation of a Cell Culture Medium

The culture medium is prepared by adding fetal bovine serum, hbFGF, hSCF, non-essential amino acids, L-glutamine and gentamicin into a DMEM culture medium with the glucose content of 2.5 g/L, wherein every 100 mL of the final culture medium contains 18% by volume percent of fetal bovine serum, 2000 ng of hbFGF, 1000 ng of hSCF, 0.1 mL of 100× non-essential amino acids, 1 mL of PBS solution containing 3% by mass percent of L-glutamine and 4000 U of gentamicin. The 100× non-essential amino acids were purchased from GIBCO Company and the article number is 11140.

### 2. Obtaining of Epidermal Tissue and Epidermal Cells:

Clinically abandoned foreskin tissue was taken and soaked in PBS solution containing 1000 U of gentamicin per mL, and then preserved at the temperature of 4° C. within 4 hours. After fully washed with the PBS to remove the blood cells, the tissue was then cut off to remove the subcutaneous loose connective tissue, followed by soaking and digesting with type II histolytic enzyme (2 U/mL) overnight so as to obtain the epidermis tissue. The epidermis tissue was cut into pieces and then digested with 0.25% by mass percent of trypsin for 30 minutes to obtain the epidermal cells. Next, the epidermis cells were washed with PBS centrifuged to remove the digestive enzyme and re-suspending in the cell culture medium provided in the present embodiment.

### 3. Culture of the Epidermal Cells

The epidermal cells obtained from each specimen were harvested into 3-4 T25 culture flasks containing the cell culture medium provided by the present embodiment, cultured in an incubator at 36±1° C. with 5±1% CO₂ and 90-100% humidity. The cell culture medium was changed every 2-3 days.

### 4. Screening and Enrichment of Human Epidermis-Derived Mesenchymal Stem Cell-Like Pluripotent Cells

When the cells reached 60-90% confluence, the cultures were digested with 0.25% trypsin/0.02% wt EDTA (1:1). The digested mesenchymal stem cell-like cells were collected and undigested cells were discarded. The collected mesenchymal stem cell-like cells were centrifuged at 1000-1500 rpm for 5 minutes and the supernatant was discarded. The cells were re-suspended by using the cell culture medium provided in the present embodiment. Collected cells were the human epidermis-derived mesenchymal stem cell-like pluripotent cells in the invention. The cells were further counted.

### 5. In-Vitro Culture of Human Epidermis-Derived Mesenchymal Stem Cell-Like Pluripotent Cell Strain

The well-counted human epidermis-derived mesenchymal stem cell-like pluripotent cells were inoculated into T25 culture flasks containing the cell culture medium provided by the present embodiment to about 1×10⁵/mL, cultured in the incubator under at 36±1° C. with 5±1% CO₂ and 90-100% humidity, and passed to a proportion of 1:3 every 1-4 days till over 30 generations. The human epidermis-derived mesenchymal stem cell-like pluripotent cell strain is therefore obtained. The cells obtained from each clinically abandoned skin specimen can be passed above 30 generations within 3 months, and at least 2×10¹⁶ new human epidermis-derived mesenchymal stem cell-like pluripotent cells can be finally obtained from about 3×10⁴ cells.

By detecting of the human epidermis-derived mesenchymal stem cell-like pluripotent cell strain provided by the present embodiment, it was found that the cell strain has mesenchymal stem cell-like phenotype and normal karyotype, and is able to express specific markers of mesenchymal stem cells and neural precursor cells, including CD73, CD90, CD105, Vimentin and Nestin, and realize no expression or low expression of the specific markers of neural terminal cells, epidermal cells, blood cells and vascular endothelial cells, including β-III tubulin, MAP-2, GFAP, CK19, CD34, CD45, CD3, CD19, CD16, CD4, CD8, CD31, VEGF-R2 and CD10. The cell strain can differentiate into neural cell-like cells and immune cell-like cells, and realize low expression or no expression of HLA-DR and low expression or moderate expression of HLA-I. After 3 months of intraperitoneal injection and subcutaneous injection of about 1×10⁷ cells into each SCID mouse, no tumors were found in the mice. This indicates that the human epidermis-derived mesenchymal stem cell-like pluripotent cell strain in the present invention can provide good biological safety.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A culture medium, wherein the culture medium is formed by adding fetal bovine serum, human basic fibroblast growth factor (hbFGF), human stem cell factor (hSCF), non-essential amino acids, L-glutamine and gentamicin into a Dulbecco's Modified Eagle's Medium (DMEM) with the glucose content of 0.5-5 g/L, and every 100 mL of the culture medium comprises 10-25% by volume percent of fetal bovine serum, 100-4000 ng of hbFGF, 10-2000 ng of hSCF, 0.1-2 mL of 100× non-essential amino acids, 0.1-2 mL of PBS solution containing 3% by mass percent of L-glutamine and 1000-8000 U of gentamicin.

2. A preparation method of human epidermis-derived mesenchymal stem cell-like pluripotent cells, comprising the following steps:
a) culturing human epidermal cells in the cell culture medium according to claim 1; and
b) then digesting said human epidermal cells with digestive juice, removing undigested human epidermal cells, and collecting digested mesenchymal stem cell-like cells so as to obtain said human epidermis-derived mesenchymal stem cell-like pluripotent cells.

3. The preparation method according to claim 2, wherein the step for obtaining said human epidermal cells in step a) comprises:
getting foreskin or other skin tissues, digesting said foreskin or other skin tissues with type II histolytic enzyme to obtain epidermis tissue, cutting said epidermis tissues into pieces, and further digesting said epidermis tissue with trypsin to obtain said human epidermal cells.

4. The preparation method according to claim 2, wherein said human epidermal cells in step a) are cultured in the cell culture medium according to claim 1 at 36±1° C. 5±1% CO₂ and 90-100% humidity, wherein the cell culture medium is changed every 2-3 days.

5. The preparation method according to claim 2, wherein step b) comprises the following steps:
when reaching 60-90% confluence, digesting said human epidermal cells with 0.25% trypsin/0.02% by mass percentage of EDTA (1:1), removing undigested human epidermal cells, and collecting digested mesenchymal stem cell-like cells so as to obtain said human epidermis-derived mesenchymal stem cell-like pluripotent cells.

6. Human epidermis-derived mesenchymal stem cell-like pluripotent cells, prepared by the preparation method according to claim 2.

7. A method for passage culturing human epidermis-derived mesenchymal stem cell-like pluripotent cells according to claim 6, wherein the method comprises the follow step:
culturing said human epidermis-derived mesenchymal stem cell-like pluripotent cells in the cell culture medium according to claim 1 at 36±1° C. 5±1% CO₂ and 90-100% humidity.
